(19) United States
(12) Patent Application Publication
(10) Pub. No.: US 2017/0108506 A1
(43) Pub. Date: Apr. 20, 2017

(54) Curve Of Life Pregnancy Test

(71) Applicants: Angela Renee Crumitie, Ocoee, FL (US); Erica Mechelle Thompson, Ocoee, FL (US)

(72) Inventors: Angela Renee Crumitie, Ocoee, FL (US); Erica Mechelle Thompson, Ocoee, FL (US)

(21) Appl. No.: 15/336,621

(22) Filed: Oct. 27, 2016

Related U.S. Application Data
(60) Provisional application No. 62/386,264, filed on Nov. 24, 2015.

(51) Int. Cl. G01N 33/68

(52) U.S. Cl. CPC G01N 33/689

(57) ABSTRACT

The Curve of Life Pregnancy Test is a newly designed pregnancy test that takes the shape of a woman which includes curves and hair, making the pregnancy test visually appealing. The pregnancy test includes a window in the stomach area that shows different results than standard pregnancy tests available on the market. If the results are positive, a baby will appear in the window. If the test is negative, a circle will appear. The test also includes a detachable spoon where the urine will be collected to provide results. The test provides more sanitation because the bottom part is shaped like a spoon, which is more likely to catch the urine and can be detached without touching the spoon. Once the results appear the spoon detaches allowing the test to become a keep sake souvenir.

### SUMMARY OF DISCLOSURE

The Curve of Life Pregnancy Test is a newly designed pregnancy test that takes the shape of a woman. The test has hair and has curves that women are likely to relate to. Starting with the oval shaped head and hair which flows off the shoulder to the right side of test. The head appears to be turned to the right to show the side of the hair. From the head extends a small portion of the neck; from the neck extends the breast area of the woman; from breast area extends the stomach area which contains a window that will show the results of the pregnancy test. If the pregnancy test is positive, the outline of a baby will appears. If the test if negative, the area will show a full circle. From the stomach extends the curve of a woman's hips; from the hips extends the bottom portion of the pregnancy test which is rectangular. From this portion and down will be where the pregnancy test detaches. The rectangular portion leads down to the circular scoop where the woman will urinate to get results. The urine scoop will then provide the results, and be ejected by pressing the top of the woman's hair in the right corner of the portion of the head. By pressing the button on the hair, the urine scoop will then eject and can be discarded, therefore turning “The Curve of Life Pregnancy Test” into a keepsake or memorabilia.

### BRIEF DESCRIPTION OF DRAWINGS

FIG. 1 is a front view of the Curve of Life pregnancy Test

FIG. 2 is a back view of the Curve of Life Pregnancy Test

FIG. 3 is a right side view of the Curve of Life Pregnancy Test

FIG. 4 is a left side view of the Curve of Life Pregnancy Test

FIG. 5 is a top view of the Curve of Life Pregnancy Test

FIG. 6 is a Bottom view of the Curve of Life Pregnancy Test

### DETAILED DESCRIPTION OF THE DRAWINGS

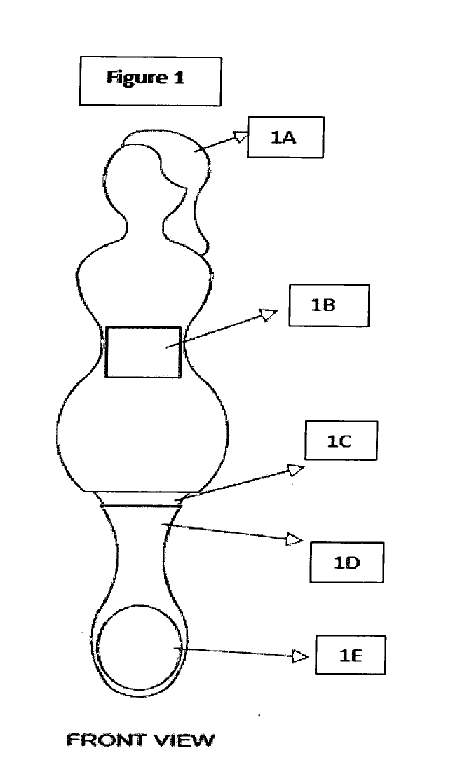

FIG. 1A illustrates an example of the hair

FIG. 1B Illustrates an example of the Results window where an baby appears if positive or an empty circle with appear if negative

FIG. 1C illustrates an example of the rectangular portion where the scoop will detach from the upper portion of the test.

FIG. 1D illustrates the outer casing of the scoop which will house the urine strip.

FIG. 1E illustrates the example of the litmus paper where the urine strip be collected. The strip is housed by the outer casing of the scoop.

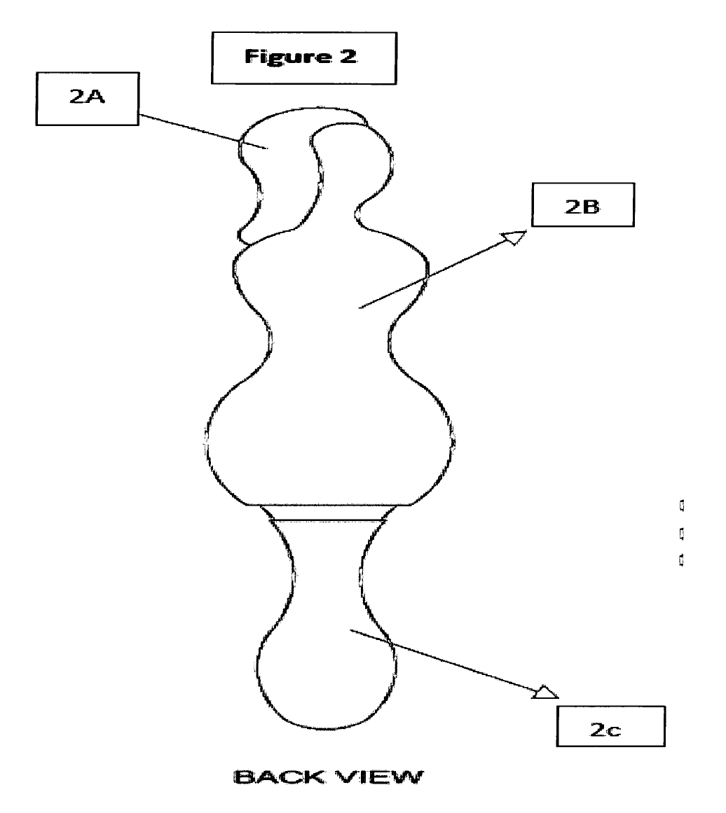

FIG. 2A illustrates an example of the back view of the hair

FIG. 2B illustrates the back of the pregnancy test which is the upper breast portion of the Curve of Life Pregnancy Test.

FIG. 2C illustrates and example of the back portion of the casing of the scoop

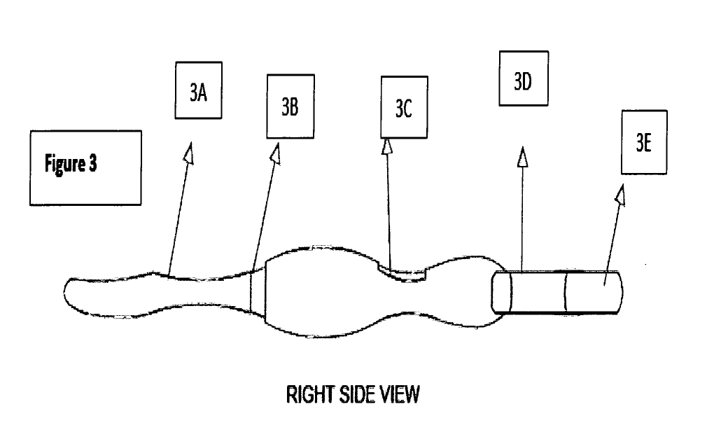

FIG. 3A illustrates the right side of the outer casing of the scoop

FIG. 3B illustrates an example of the right side of the rectangular portion that detaches from the upper portion of the pregnancy test.

FIG. 3C illustrates the right side view of the results window.

FIG. 3D illustrates the right side view of the bottom portion of the hair

FIG. 3E Illustrates the example of the right view of the eject button. Once the ejection button is pushed the scoop will detach from the rectangular portion on the bottom of the test.

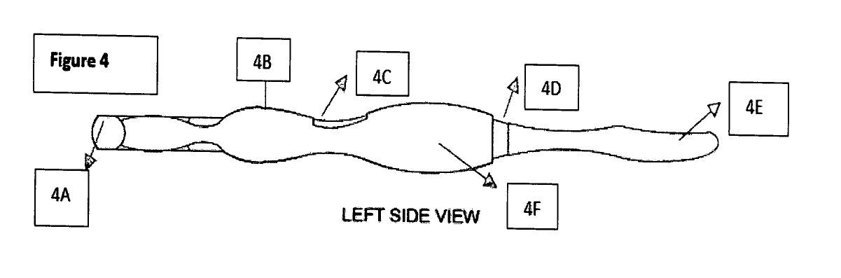

FIG. 4A Illustrates the left view of the eject button in the top portion of the hair

FIG. 4B illustrates the left view of the breast portion of the Curve of Life Pregnancy Test

FIG. 4C Illustrates the left top view of the results window

FIG. 4D illustrates the left view of the Rectangular portion which is where the scoop detaches

FIG. 4E illustrates the left view of the outer casing of the scoop

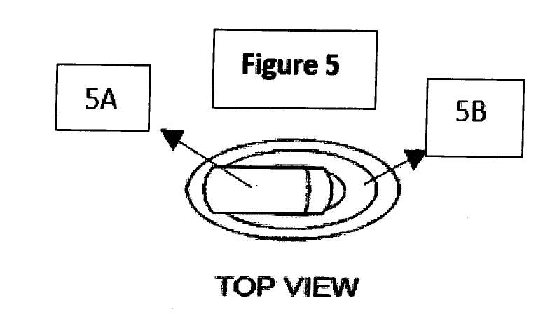

FIG. 5A illustrates the top view of the ejection button which will be pressed in order to detach the scoop

FIG. 5B illustrates the top view of the hair

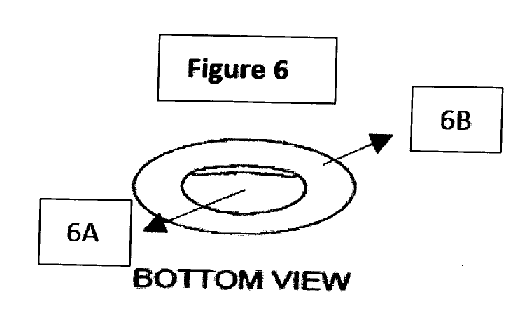

FIG. 6A illustrates the bottom view of the litmus paper that will be housed inside of the scoop

FIG. 6B illustrates the bottom view of the outer casing of the scoop

1. I claim the curve of life pregnancy test which is shaped like a woman and is the only pregnancy test that provides a detachable strip.
I claim The Curve of Life Pregnancy Test which is made to be a keepsake once the detachable strip is disposed of.
I claim The Curve of Life Pregnancy Test which shows an outline of a baby once the results show the test is positive. .
